(19) United States
(12) Patent Application Publication
(10) Pub. No.: US 2025/0268567 A1
(43) Pub. Date: Aug. 28, 2025

(54) ULTRASONIC ENDOSCOPE

(71) Applicant: FUJIFILM Corporation, Tokyo (JP)

(72) Inventors: Yasuhiko MORIMOTO, Ashigarakami-gun (JP); Kazushi FURUKAWA, Ashigarakami-gun (JP); Karin HATTORI, Ashigarakami-gun (JP); Shozo IYAMA, Ashigarakami-gun (JP)

(73) Assignee: FUJIFILM Corporation, Tokyo (JP)

(21) Appl. No.: 19/060,759

(22) Filed: Feb. 23, 2025

(30) Foreign Application Priority Data
Feb. 28, 2024 (JP) 2024-028457

(51) Int. Cl. A61B 8/12; A61B 1/00; A61B 8/00; G01S 7/52

(52) U.S. Cl. CPC A61B 8/12; A61B 1/00071; A61B 1/00163; A61B 8/4444; G01S 7/5202

(57) ABSTRACT

An ultrasonic endoscope includes: a tubular member containing a metal; an ultrasonic oscillator that is provided along an outer peripheral surface of the tubular member, and a backing material layer that is provided between the ultrasonic oscillator and the tubular member, and the backing material layer containing at least one kind of a polyurea resin, an epoxy resin having a polyurethane structure, or an epoxy resin having a polyetheramine structure.

### CROSS-REFERENCE TO RELATED APPLICATIONS

The present application claims priority under 35 U.S.C. § 119 to Japanese Patent Application No. 2024-028457 filed on Feb. 28, 2024. The above application is hereby expressly incorporated by reference, in its entirety, into the present application.

### BACKGROUND OF THE INVENTION

### 1. Field of the Invention

The present invention relates to an ultrasonic endoscope.

### 2. Description of the Related Art

JP2022-124502A, WO2018/003737A, and JP2022-175241A describe an ultrasonic endoscope including an ultrasonic oscillator array in which a plurality of ultrasonic oscillators are arranged in a cylindrical shape.

### SUMMARY OF THE INVENTION

An object of the present disclosure is to provide an ultrasonic endoscope with improved durability.

An ultrasonic endoscope of one embodiment according to the technology of the present disclosure comprises: a tubular member that is configured to include a metal; an ultrasonic oscillator that is provided along an outer peripheral surface of the tubular member; and a backing material layer that is provided between the ultrasonic oscillator and the tubular member, in which the backing material layer is configured to include at least one kind of a polyurea resin, an epoxy resin having a polyurethane structure, or an epoxy resin having a polyetheramine structure.

According to the technology of the present disclosure, a durability can be improved.

### BRIEF DESCRIPTION OF THE DRAWINGS

FIG. 1 is a schematic configuration diagram showing an endoscope apparatus using an ultrasonic endoscope, which is an aspect of the technology of the present disclosure.

FIG. 2 is a partially enlarged perspective view showing an appearance of an example of a distal end part of the ultrasonic endoscope shown in FIG. 1.

FIG. 3 is a longitudinal cross-sectional view taken along an axis of the distal end part of the ultrasonic endoscope shown in FIG. 2.

FIG. 4 is a partially enlarged view of FIG. 3.

### DESCRIPTION OF THE PREFERRED EMBODIMENTS

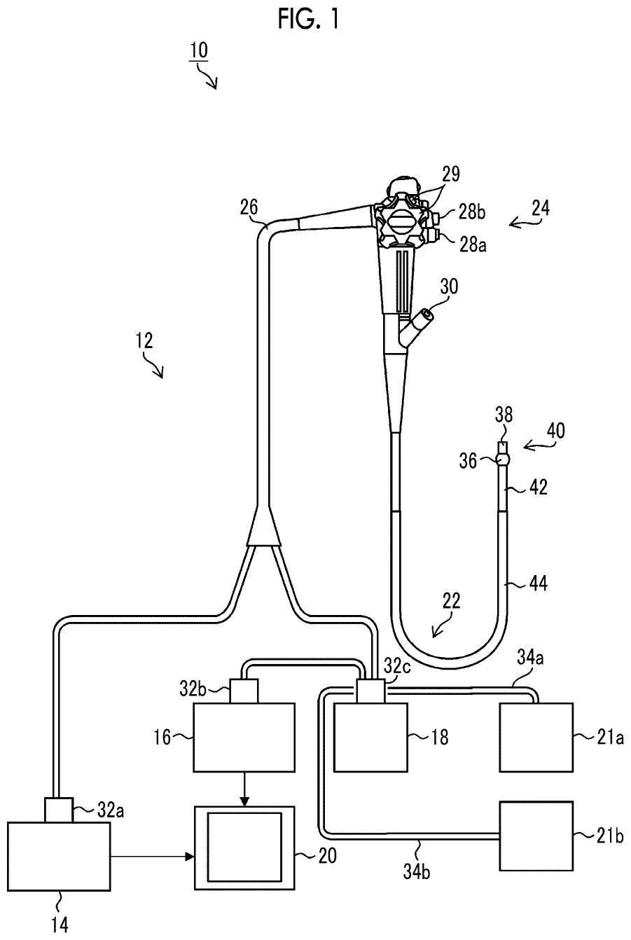

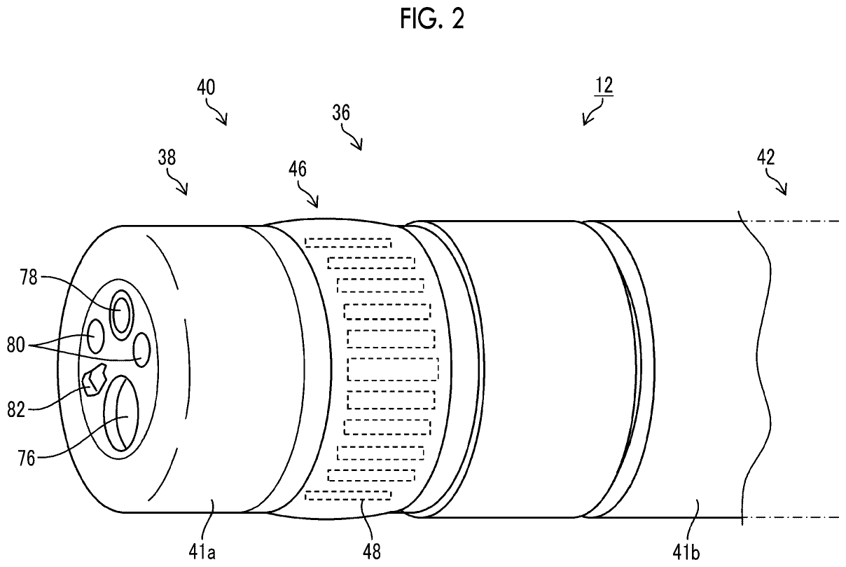

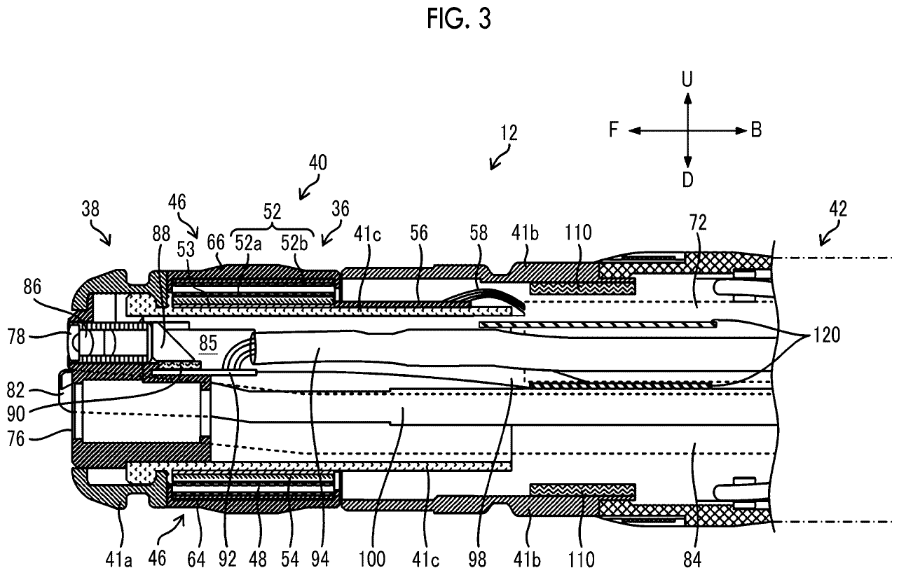

FIG. 1 is a schematic configuration diagram showing an endoscope apparatus 10 that uses an ultrasonic endoscope 12 according to an aspect of the technology of the present disclosure. FIG. 2 is a partially enlarged perspective view showing an appearance of an example of a distal end part of the ultrasonic endoscope 12 shown in FIG. 1. FIG. 3 is a longitudinal cross-sectional view taken along an axis of the distal end part of the ultrasonic endoscope 12 shown in FIG. 2.

As shown in FIG. 1, the endoscope apparatus 10 comprises an ultrasonic endoscope 12, an ultrasound processor device 14 that generates an ultrasound image, an endoscope processor device 16 that generates an endoscopic image, a light source device 18 that supplies illumination light for illuminating an inside of a body cavity to the ultrasonic endoscope 12, a monitor 20 that displays the ultrasound image, the endoscopic image, and the like, a water supply tank 21a that stores cleaning water and the like, and a suction pump 21b that suctions an object to be suctioned in the body cavity.

The ultrasonic endoscope 12 has an insertion part 22 that is inserted into a body cavity of a subject, an operating part 24 that is consecutively provided in a proximal end part of the insertion part 22 and is used by an operator to perform an operation, and a universal cord 26 that has one end connected to the operating part 24.

In the operating part 24, an air/water supply button 28a that opens and closes an air/water supply pipe line (not shown) from the water supply tank 21a, and a suction button 28b that opens and closes a suction pipe line (not shown) from the suction pump 21b are provided side by side. In the operating part 24, a pair of angle knobs 29 and a treatment tool insertion port 30 are provided.

In the other end part of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasonic endoscope 12 is attachably and detachably connected to the ultrasound processor device 14, the endoscope processor device 16, and the light source device 18 via the connectors 32a, 32b, and 32c, respectively. The connector 32c comprises an air/water supply tube 34a that is connected to the water supply tank 21a, and a suction tube 34b that is connected to the suction pump 21b.

The insertion part 22 includes, in order from a distal end side, a distal end part 40 formed of a hard member and having an ultrasonic observation part 36 and an endoscope observation part 38, a bendable part 42 that is consecutively provided on a proximal end side of the distal end part 40, and a soft part 44 that connects a proximal end side of the bendable part 42 and a distal end side of the operating part 24. The bendable part 42 is formed by connecting a plurality of bending pieces (angle rings) and is configured to be bendable. The soft part 44 is elongated and long and has flexibility.

The ultrasound processor device 14 generates and supplies an ultrasound signal for causing a plurality of ultrasonic oscillators 48 of an ultrasound transmission/reception section 46 (see FIG. 2) of the ultrasonic observation part 36, which will be described later, to generate ultrasonic waves. A vibration frequency of the ultrasonic oscillator 48 used in the ultrasonic endoscope 12 preferably has a center frequency of 5 MHz or more and 12 MHz or less. The ultrasound processor device 14 receives and acquires an echo signal reflected from an observation target site irradiated with the ultrasonic wave, by the ultrasonic oscillator 48 and executes various kinds of signal processing on the acquired echo signal to generate an ultrasound image. The generated ultrasound image is displayed on the monitor 20.

The endoscope processor device 16 receives and acquires an image signal acquired from the observation target site illuminated with illumination light from the light source device 18 in the endoscope observation part 38, and executes various kinds of signal processing and image processing on the acquired image signal to generate an endoscopic image. The generated endoscopic image is displayed on the monitor 20.

In order to image an observation target site inside a body cavity using the endoscope observation part 38 to acquire an image signal, the light source device 18 generates illumination light, such as white light including light of three primary colors of red light, green light, and blue light or light of a specific wavelength. The illumination light propagates through a light guide (not shown) and the like in the ultrasonic endoscope 12 and is emitted from the endoscope observation part 38, and the observation target site inside the body cavity is illuminated with the illumination light.

Next, a configuration of the distal end part 40 will be described with reference to FIGS. 2 and 3. FIG. 3 shows a distal end direction F from the proximal end side toward the distal end side and a proximal end direction B from the distal end side to the proximal end side as directions in the insertion part 22 of the ultrasonic endoscope 12. The distal end direction F and the proximal end direction B will also be referred to as an axial direction of the insertion part 22. FIG. 3 shows an upward direction U and a downward direction D which is opposite to the upward direction U, as a radial direction (direction perpendicular to the axial direction) of the insertion part 22. As shown in FIG. 2, in the distal end part 40 of the ultrasonic endoscope 12, the ultrasonic observation part 36 for acquiring the ultrasound image is provided on the proximal end side, and the endoscope observation part 38 for acquiring the endoscopic image is provided on the distal end side.

The distal end part 40 of the ultrasonic endoscope 12 comprises a cap-shaped distal end component 41a that covers a portion of the endoscope observation part 38 on the distal end side, a tubular proximal end-side ring 41b that is disposed on the proximal end side of the ultrasonic observation part 36 on the proximal end side, and a metal ring 41c (see FIG. 3). Here, the distal end component 41a and the proximal end-side ring 41b are made of a hard member, such as hard resin, and serve as an exterior member. The metal ring 41c is disposed on an inner side of the exterior member. The metal ring 41c is a tubular member configured to include a metal such as stainless steel (Steel Use Stainless (SUS)) or aluminum, and preferably has a cylindrical shape. A shape of the metal ring 41c does not need to be a perfect cylinder, and may be partially cut out.

The endoscope observation part 38 includes a treatment tool outlet port 76, an observation window 78, an illumination window 80, a cleaning nozzle 82, and the like, which are provided on a distal end surface thereof.

The ultrasonic observation part 36 includes the ultrasound transmission/reception section 46 supported by an outer peripheral surface of the metal ring 41c. The ultrasound transmission/reception section 46 comprises a plurality of the ultrasonic oscillators 48 that are provided on an outer periphery of the metal ring 41c along the outer peripheral surface thereof side by side, a backing material layer 54 that is provided between the plurality of ultrasonic oscillators 48 and the metal ring 41c, an interlayer 53 that is provided between the backing material layer 54 and the metal ring 41c, an electrode part 52 that comprises individual electrodes 52a corresponding to the plurality of ultrasonic oscillators 48 and a common electrode 52b common to the plurality of ultrasonic oscillators 48, a flexible printed substrate 56 to which each individual electrode 52a is connected, an acoustic matching layer 64 having a substantially cylindrical shape that is laminated on the plurality of ultrasonic oscillators 48, and an acoustic lens 66 having a substantially cylindrical shape that is laminated on the acoustic matching layer 64.

As shown in FIG. 2, the ultrasonic oscillators 48 are an array of a plurality of channels, for example, 48 to 192 channels (CH), consisting of a plurality of, for example, 48 to 192 rectangular parallelepiped ultrasonic oscillators 48 arranged in a cylindrical shape.

In the ultrasound transmission/reception section 46, a plurality of the ultrasonic oscillators 48 are arranged at a predetermined pitch in a circumferential direction as in the example shown in the drawing. As described above, the ultrasonic oscillators 48 constituting the ultrasound transmission/reception section 46 are arranged at equal intervals in a cylindrical shape with an axis of the distal end part 40 as a center. Further, each ultrasonic oscillator 48 is sequentially driven based on a driving signal input from the ultrasound processor device 14. Thus, radial electronic scanning is performed with a range in which the ultrasonic oscillators 48 are arranged, as a scanning range.

The backing material layer 54 is composed of a layer of a member made of a backing material. The backing material layer 54 has a role of mechanically and flexibly supporting the plurality of ultrasonic oscillators 48 and attenuating ultrasonic waves propagated to the backing material layer 54 side among ultrasound signals oscillated from the plurality of ultrasonic oscillators 48 or reflected and propagated from an observation target.

As shown in FIG. 3, the flexible printed substrate 56 that is attached to a side surface of the backing material layer 54 on the proximal end side is electrically connected to each individual electrode 52a of the electrode part 52 on one hand, and is wire-connected to a plurality of coaxial cables 58 of a signal line bundle 72 on the other hand. In this way, the individual electrode 52a of the ultrasonic oscillator 48 and the coaxial cable 58 are electrically connected, and each ultrasonic oscillator 48 and the signal line bundle 72 are electrically connected.

The ultrasonic endoscope 12 comprises a bracket 120 that supports the signal line bundle 72 at the distal end part 40 and extends along the signal line bundle 72, and a bracket support member 110 that supports the bracket 120.

At least one of the bracket 120 or the bracket support member 110 is preferably made of a metal. By using the metal for the bracket 120 or the bracket support member 110, a thickness thereof can be reduced. Examples of a metal material include SUS and aluminum. It is preferable that both the bracket 120 and the bracket support member 110 are made of a metal.

The acoustic lens 66 constitutes an exterior body of the distal end part 40 together with the distal end component 41a and the proximal end-side ring 41b. A distal end-side portion of the metal ring 41c with respect to the ultrasound transmission/reception section 46 is fixed to an inside of the distal end component 41a by fitting or the like. A proximal end-side portion of the metal ring 41c with respect to the ultrasound transmission/reception section 46 is disposed inside the proximal end-side ring 41b in a state in which a gap is formed between the proximal end-side portion of the metal ring 41c and an inner peripheral surface of the proximal end-side ring 41b.

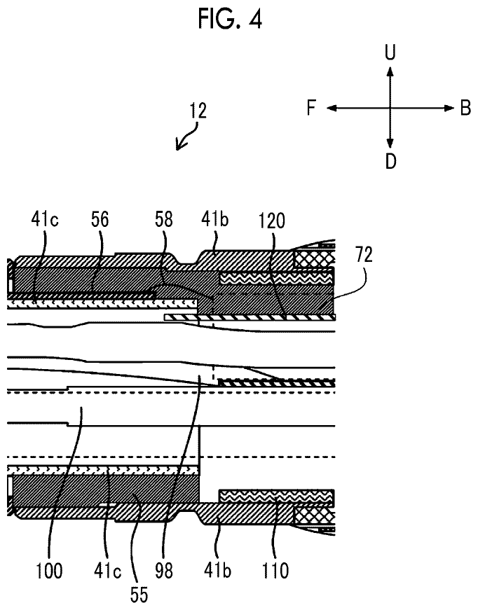

As described above, the distal end component 41a and the proximal end-side ring 41b constitute an accommodation portion that accommodates the metal ring 41c. A periphery of the proximal end-side portion of the metal ring 41c, for example, a portion between an outer peripheral surface of the proximal end-side portion of the metal ring 41c and the inner peripheral surface of the proximal end-side ring 41b is filled with a filler 55 as shown in the enlarged view of FIG. 4. The filler 55 is provided to restrict a movement of an accommodated object inside the proximal end-side ring 41b and to prevent the accommodated object from being damaged. As a material of the filler 55, an epoxy resin, a silicone resin, a urethane resin, a urea resin, or the like can be used.

The filler 55 preferably has a viscosity of less than 100 Pa·sec at a shear rate of 0.01/sec in a non-cured state. In this way, in a case where the inside of the proximal end-side ring 41b is filled with the filler 55, it is possible to prevent the filler 55 from entering a region inside the distal end part 40 that is not intended to be filled. In order to realize the above-described shear rate, for example, a filler including a resin as a base material and including a thixotropic agent such as fumed silica as an additive may be used as the filler 55. By including the thixotropic agent, a thixotropic property is improved, and thus the viscosity of the filler before curing can be increased. The thixotropic property is preferably less than 100 Pa·sec and more preferably less than 20 Pa·sec at a shear rate of 10/sec.

The backing material layer 54 reduces stress in a case where a thermal load is applied to the ultrasonic endoscope 12, thereby preventing breakage or the like and increasing a durability. Specifically, the backing material layer 54 is configured to include at least one kind of a polyurea resin, an epoxy resin having a polyurethane structure, or an epoxy resin having a polyetheramine structure, and thus can sufficiently reduce the stress in a case where a thermal load is applied. From the viewpoint of further improving the workability, it is preferable that the resin included in the backing material layer 54 includes a polyurea resin.

As the resin included in the backing material layer 54, a resin having a loss tangent of 0.06 or more in a range of 0° C. to 50° C. and a loss tangent of less than 1.50 in a range of −20° C. to 110° C. can be preferably used. It is preferable that a storage elastic modulus of the backing material layer 54 in a range of 0° C. to 50° C. is 1,000 MPa or more in a case where the content of the resin in the backing material layer 54 is 25% to 50% by volume. A thickness of the backing material layer 54 used in the ultrasonic endoscope 12 is preferably 0.5 mm or more and 1.5 mm or less.

Hereinafter, details of the preferred resin included in the backing material layer 54 will be described.

### Polyurea Resin

The polyurea resin can be obtained by a reaction between a polyisocyanate compound and a polyamine compound.

As the polyisocyanate compound, any polyisocyanate compound having two or more isocyanato groups can be used without particular limitation. The polyisocyanate compound may be any of an aliphatic isocyanate compound (a compound in which an isocyanato group is bonded to an aliphatic chain or an aliphatic ring) or an aromatic isocyanate compound (a compound in which an isocyanato group is bonded to an aromatic ring), or may be a mixture thereof. The polyisocyanate compound may have a ring structure. From the viewpoint of low reactivity and a long pot life during production of a cured substance, an aliphatic polyisocyanate compound is preferable as the polyisocyanate compound, and from the viewpoint of further improving ultrasonic wave attenuation properties, it is preferable that the polyisocyanate compound includes an aliphatic polyisocyanate compound having an aromatic ring and an aromatic polyisocyanate compound.

The polyamine compound can be used without particular limitation as long as it is a polyamine compound having two or more amino groups, and a polyamine compound generally used as a curing agent for an epoxy resin is preferably used. The polyamine compound may be any of an aliphatic polyamine compound (a chain-like aliphatic polyamine compound in which an amino group is bonded to an aliphatic chain or a cyclic aliphatic polyamine compound in which an amino group is bonded to an aliphatic ring) or an aromatic polyamine compound (a compound in which an amino group is bonded to an aromatic ring), and may be a mixture thereof. Among these, an aliphatic polyamine compound is preferable because it has excellent reactivity. The polyamine compound may have a ring structure. In addition to a nitrogen atom, a heteroatom such as an oxygen atom may be included. From the viewpoint of further improving the ultrasonic wave attenuation properties and the workability, the polyamine compound preferably includes an aliphatic polyamine compound having an aromatic ring and a chain-like aliphatic polyamine compound having no aromatic ring.

### Epoxy Resin Having Polyurethane Structure

The epoxy resin having a polyurethane structure can be used without particular limitation as long as it is an epoxy resin having a polyurethane structure and an epoxy group. As a commercially available epoxy resin having a polyurethane structure, for example, an epoxy resin having a polyurethane structure with a number average molecular weight of 200 to 20,000 is generally used. A viscosity of the epoxy resin having a polyurethane structure at 25° C. is not particularly limited, and is, for example, preferably 200 to 200,000 mPa·sec and more preferably 600 to 30,000 mPa·sec. The viscosity is a value measured under conditions of 25° C. and a shear rate of 0.01/sec.

As the curing agent for reacting with the epoxy resin having a polyurethane structure, a polyamine or an acid anhydride can be used, but it is preferable to use a polyamine as the curing agent. The polyamine compound to be reacted with the epoxy resin having a polyurethane structure can be used without particular limitation as long as it is a polyamine compound having two or more amino groups, and a polyamine compound generally used as a curing agent for an epoxy resin is preferably used.

### Epoxy Resin Having Polyetheramine Structure

As the epoxy resin having a polyetheramine structure, a reaction cured product of an epoxy resin and a polyamine compound having two or more amino groups can be used without particular limitation as long as it has a polyether structure.

The epoxy resin having a polyetheramine structure can be obtained by any of a reaction between an epoxy resin having a polyether structure and a polyamine compound having no polyether structure, a reaction between an epoxy resin having no polyether structure and a polyamine compound having a polyether structure, or a reaction between an epoxy resin having a polyether structure and a polyamine compound having a polyether structure. In general, the polyether structure of the reaction cured product obtained in this way is generally a polyether structure having a number average molecular weight of 200 to 6,000.

Among these, a reaction cured product of an epoxy resin having a polyether structure and a polyamine compound having no polyether structure, or a reaction cured product of an epoxy resin having no polyether structure and a polyamine compound having a polyether structure is preferable, and from the viewpoint of exhibiting a more preferable viscosity as a curable resin composition, a reaction cured product of an epoxy resin having no polyether structure and a polyamine compound having a polyether structure is more preferable.

As a commercially available epoxy resin having a polyether structure, for example, an epoxy resin having a polyether structure with a number average molecular weight of 200 to 6,000 is generally used, and specific examples thereof include the following epoxy resins. From the viewpoint of excellent mechanical strength, it is preferable that the epoxy resin has a bisphenol structure. Examples of the epoxy resin having a polyether structure include a bisphenol A epoxy resin, a bisphenol F epoxy resin, a bisphenol E epoxy resin, and a novolac epoxy resin. From the viewpoint of excellent mechanical strength of a cured product, a bisphenol A epoxy resin is preferable.

The polyamine compound having a polyether structure can be used without particular limitation as long as it is a polyamine compound having two or more amino groups, and a polyamine compound generally used as a curing agent for an epoxy resin is preferably used. As a commercially available polyamine compound having a polyether structure, for example, a polyamine compound having a polyether structure with a number average molecular weight of 200 to 6,000 is generally used.

### Content of Resin in Backing Material Layer

A content of a resin in the backing material layer 54 is 25% to 50% by volume, and preferably 30% to 50% by volume. A content of a reaction cured product of at least one kind of the polyurea resin, the epoxy resin having a polyurethane structure, or the epoxy resin having a polyetheramine structure, which are resins included in the backing material layer 54, is not particularly limited as long as the effects of the technology of the present disclosure are exhibited. For example, the content can be 15% by volume or more, and is preferably 20% by volume or more, more preferably 30% by volume or more, still more preferably 50% by volume or more, and particularly preferably 70% by volume or more. It is also preferable that all the resins included in the backing material layer 54 are composed of at least one kind of the polyurea resin, the epoxy resin having a polyurethane structure, or the epoxy resin having a polyetheramine structure.

The backing material layer 54 is preferably configured using at least one kind of the polyurea resin, the epoxy resin having a polyurethane structure, or the epoxy resin having a polyetheramine structure as a base material, and including a heat radiation filler. The backing material layer 54 includes thermally conductive particles as the heat radiation filler, and thus a thermal conductivity can be increased. By increasing the thermal conductivity of the backing material layer 54, heat generated in the ultrasound transmission/reception section 46 can be transmitted to a heat radiation structure (not shown), and accumulation of the heat in the distal end part 40 can be prevented. As a result, a thermal load on the backing material layer 54 is reduced, and stress due to the thermal load can be further reduced. In addition, even in a case where a difference between a thermal expansion coefficient of the base material of the backing material layer 54 and a thermal expansion coefficient of the metal ring 41c is somewhat large, the stress applied to the base material can be reduced by heat radiation of the heat radiation filler.

As long as the thermally conductive particles have thermally conductive properties, any of inorganic particles or organic particles may be used. In order to increase the thermally conductive properties of the backing material layer 54, a thermal conductivity per unit weight of the backing material layer 54 is preferably 30 W/m·K or more and more preferably 60 W/m·K or more. Since the ultrasonic endoscope 12 is inserted into a body, it is preferable that the thermally conductive particles are a safe material having no toxicity or the like and are stable against a use environment such as hygroscopicity. In addition, in order to increase the attenuation properties, it is preferable that a density of the thermally conductive particles is high. Since the thermally conductive particles are disposed in the vicinity of a circuit, a material having low or no conductivity that does not cause a short circuit failure is preferable.

A shape of the thermally conductive particle is not particularly limited, and various shapes such as an amorphous shape, a spherical shape, a fibrous shape, a branched fibrous shape, and a flat plate shape are used. In a case where the shape is a spherical shape, it is possible to increase a filling rate, which is preferable. In a case where the shape is an anisotropic shape such as a fibrous shape or a flat plate shape, it is possible to increase a particle contact and improve heat radiation properties, which is preferable. In a case of the amorphous particles, the ultrasonic waves can be randomly reflected, which is preferable from the viewpoint of improving the ultrasonic wave attenuation properties of the backing material layer 54.

Examples of the thermally conductive particles include aluminum oxide, tungsten oxide, silicon carbide, tungsten carbide, silicon nitride, boron nitride, and aluminum nitride. In particular, from the viewpoint of high thermal conductivity and high insulating properties, a nitride is preferable. The thermally conductive particles may include one kind of these thermally conductive materials or may include two or more kinds thereof. A surface of the thermally conductive particle may be subjected to a surface treatment in order to easily disperse in the resin.

A particle diameter of the thermally conductive particle is not particularly limited. From the viewpoint of maintaining the mechanical strength of the backing material layer 54 high while suppressing the viscosity of the curable resin composition included in the backing material layer 54 to be low, for example, the particle diameter of the thermally conductive particle is preferably 1 to 300 μm, more preferably 5 to 100 μm, and still more preferably 8 to 30 μm. The “particle diameter” of the thermally conductive particle is a number average particle diameter.

A proportion of the thermally conductive particles in a total amount of components other than the above-described resins in the backing material layer 54 is preferably 50% by volume or more, more preferably 60% by volume or more, and still more preferably 65% by volume or more. It is also preferable that all the components other than the above-described resins in the backing material layer 54 are thermally conductive particles. For example, the content of the thermally conductive particles in the backing material layer 54 is preferably 30% to 60% by volume, more preferably 30% to 55% by volume, and still more preferably 30% to 50% by volume.

The backing material layer 54 may contain other components in addition to the above-described resins and thermally conductive particles. Hollow particles may be included as the other components. By including the hollow particles, the ultrasonic wave attenuation properties can be further improved. As the hollow particles, hollow particles commonly used for exhibiting the effect of improving acoustic wave attenuation properties or ultrasonic wave attenuation properties can be used without particular limitation. Any of hollow glass particles or hollow resin particles may be used, and it is preferable to use hollow resin particles.

Preferred examples of the hollow particles include glass balloons, hollow silica, cenolite, phenolic resin microballoons, urea resin microballoons, polymethyl methacrylate balloons, and thermal expansion microcapsules. One kind of the hollow particles may be used alone, or two or more kinds of the hollow particles may be used in combination. In the present specification, in a case where two or more kinds of hollow particles are contained, the content of the hollow particles means a total amount thereof.

A particle diameter of the hollow particle is not particularly limited. From the viewpoint of maintaining the mechanical strength of the backing material layer 54 high while suppressing the viscosity of the curable resin composition to be low, for example, the particle diameter of the hollow particle is preferably 1 to 300 μm, more preferably 5 to 100 μm, and still more preferably 20 to 80 μm. The “particle diameter” of the hollow particle is synonymous with the “particle diameter” of the thermally conductive particle described above. That is, the “particle diameter” of the hollow particle is a number average particle diameter.

The other components may include a dispersant, a diluent, a colorant, a viscosity adjuster, a plasticizer, a curing accelerator, and the like. The content of the other components in the backing material layer 54 can be, for example, 10% to 20% by volume.

Examples of a preferred form of the backing material layer 54 include a form in which a resin including at least one kind of a polyurea resin, an epoxy resin having a polyurethane structure, or an epoxy resin having a polyetheramine structure, and thermally conductive particles are included, the resin has the above-described specific loss tangent, the backing material layer has the above-described specific storage elastic modulus, and the above-described hollow particles are included. In this form, regarding a content of each component in the backing material layer 54, the content of the resin is 25 to 50% by volume, and preferably 30 to 50% by volume. The content of the thermally conductive particles is preferably 30% to 60% by volume, more preferably 30% to 55% by volume, and still more preferably 30% to 50% by volume. The content of the hollow particles is preferably 10% to 20% by volume.

The backing material layer 54 is preferably formed of a curable resin composition. The curable resin composition includes the thermally conductive particles and a resin component which is any of a combination of a polyisocyanate compound and a polyamine compound, a combination of an epoxy resin having a polyurethane structure and a polyamine compound, or a combination of an epoxy resin and a polyamine compound, in which at least one of the epoxy resin or the polyamine compound has a polyether structure.

### Method of Manufacturing Backing Material Layer

The curable resin composition constituting the backing material layer 54 can be prepared by a routine method. For example, the curable resin composition can be obtained by kneading the above-described thermally conductive particles, the resin component including at least one kind of a polyurea resin, an epoxy resin having a polyurethane structure, or an epoxy resin having a polyetheramine structure, and appropriately other components, as the components constituting the curable resin composition, with a kneading device such as a planetary centrifugal mixer, a kneader, a pressurization kneader, a Banbury mixer (continuous kneader), or two rolls. A mixing order of each component is not particularly limited. Kneading conditions are not particularly limited, and it is sufficient that the thermally conductive particles are dispersed in the resin component.

By curing the curable resin composition obtained in this way, the backing material layer 54 can be obtained. The curing conditions can be adjusted according to a chemical reaction of the resin component contained in the curable resin composition, and for example, the backing material layer 54 can be obtained by heating and curing at a specific temperature for a certain period of time.

The shape of the backing material layer 54 is not particularly limited. For example, the backing material layer may have a preferred shape by a mold during curing, or a desired backing material may be obtained by preparing a sheet-shaped backing material and cutting this backing material by die cutting or the like. Since the backing material layer 54 of the present disclosure has excellent workability, it is possible to manufacture a desired backing material layer while suppressing an occurrence of deformation, breakage, and the like even in a case of performing dicing work into a desired shape at a pitch of μm order.

The interlayer 53 is provided to prevent ions generated from the metal included in the metal ring 41c from coming into contact with the backing material layer 54. The metal ring 41c generates ions that can be a factor of deterioration of the backing material layer 54 made of the above-described material by a hydrogen peroxide gas used for sterilization. The presence of the interlayer 53 prevents the ions from reaching the backing material layer 54, and thus the backing material layer 54 can be prevented from deteriorating.

It is preferable that the interlayer 53 is thin for a reduction in diameter of the distal end part 40. For example, it is preferable that the interlayer 53 is formed by coating the outer peripheral surface of the metal ring 41c with a material by application, attachment, or the like, or the interlayer 53 is formed by performing a surface treatment on the outer peripheral surface of the metal ring 41c. As the interlayer 53, a tape obtained by applying a heat-resistant silicone-based pressure sensitive adhesive to a polyimide film base, a diamond-like carbon (DLC) coating, a silicon coating, a Parylene coating, ABEL BLACK (registered trademark), or the like can be employed. It is more preferable that the interlayer 53 is formed of a material having high withstand voltage performance. In addition, by providing the interlayer 53 also on an end surface of the backing material layer 54 in the axial direction, the arrival of ions at the backing material layer 54 can be further suppressed.

The metal ring 41c and the backing material layer 54 are fixed via the interlayer 53. Specifically, an adhesive is applied to a surface of the interlayer 53 formed by coating or the like on an outer surface of the metal ring 41c, and the interlayer 53 and the backing material layer 54 are fixed by the adhesive. As described above, the metal ring 41c and the backing material layer 54 are fixed via the interlayer 53, so that even in a case where there is a large difference in thermal expansion coefficient between the metal ring 41c and the backing material layer 54, the stress applied to the backing material layer 54 can be reduced.

A balloon (not shown) into which an ultrasound transmission medium (for example, water, oil, or the like) covering the ultrasonic observation part 36 is injected may be attachably and detachably mounted on the distal end part 40.

As shown in FIG. 3, in the distal end part 40, an observation system unit 85 is disposed behind (on the proximal end side of) the observation window 78. The observation system unit 85 includes, for example, an objective lens 86, a prism 88, an imaging element 90, a substrate 92, and a signal cable 94. A distal end-side portion of the observation system unit 85 is inserted into the metal ring 41c. The observation system unit 85 constitutes an imaging module.

The reflected light of the observation target site incident from the observation window 78 is captured by the objective lens 86. An optical path of the captured reflected light is bent at a right angle by the prism 88, and the reflected light is imaged on an imaging surface of the imaging element 90. The imaging element 90 photoelectrically converts the reflected light of the observation target site that has transmitted through the observation window 78, the objective lens 86, and the prism 88 and imaged on the imaging surface, to output an image signal.

The imaging element 90 is mounted on the substrate 92. A circuit pattern (not shown) electrically connected to the imaging element 90 is formed in the substrate 92. The circuit pattern comprises a plurality of electrodes in an end part, and a plurality of signal cables 94 are connected to the plurality of electrodes, respectively. The plurality of signal cables 94 are connected to the endoscope connector 32b (see FIG. 1). The endoscope connector 32b is connected to the endoscope processor device 16.

An emission end of a light guide 98 is connected to the illumination windows 80 (see FIG. 2). An incidence end of the light guide 98 is connected to the light source device 18 via the universal cord 26. Illumination light emitted from the light source device 18 propagates through the light guide 98, and a site to be observed is irradiated with the illumination light from the illumination windows 80.

In order to clean surfaces of the observation window 78 and the illumination windows 80, the cleaning nozzle 82 jets air or cleaning water from the water supply tank 21a toward the observation window 78 and the illumination windows 80 through an air/water supply channel 100 in the ultrasonic endoscope 12. A treatment tool channel 84 is connected to the treatment tool outlet port 76.

### EXAMPLES

Hereinafter, examples of the backing material layer 54 of the technology of the present disclosure will be described, but the backing material layer 54 is not limited to results thereof.

### <1> Preparation of Composition for Backing Material Layer

A composition for a backing material layer (curable resin composition) having the following composition was prepared.

### Polyurea Resin

A composition for a backing material layer was prepared by mixing 2.5 parts of metaxylene diisocyanate (manufactured by Tokyo Chemical Industry Co., Ltd.) as a polyisocyanate, 45 parts of a resin composition mixed at a ratio of 2 parts of ELASMER 250P (manufactured by Kumiai Chemical Industry Co., Ltd.) and 8 parts of ELASMER 650P (manufactured by Kumiai Chemical Industry Co., Ltd.) as a polyamine, and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### Epoxy Resin Having Polyurethane Structure

A composition for a backing material layer was prepared by mixing 10 parts of ADEKA RESIN EPU-11F (manufactured by ADEKA Corporation) as an epoxy resin having a polyurethane structure, 45 parts of a resin composition mixed at a ratio of 0.6 parts of 2,2,4-trimethylhexamethylenediamine (manufactured by Tokyo Chemical Industry Co., Ltd.) and 1.0 part of Gaskamine-328 (manufactured by Mitsubishi Gas Chemical Company, Inc.), and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### Epoxy Resin Having Polyetheramine Structure

A composition for a backing material layer was prepared by mixing 10 parts of jER828 (manufactured by Mitsubishi Chemical Corporation) as a bisphenol A epoxy resin, 45 parts of a resin composition mixed at a ratio of 4.5 parts of JEFFAMINE D400 (manufactured by Huntsman Corporation) and 6.0 parts of JEFFAMINE D2000 (manufactured by Huntsman Corporation) as a bifunctional polyether polyamine, and 25 parts of tungsten carbide particles (WC-100S (manufactured by A.L.M.T. Corp.)) and 15 parts of silicon carbide particles (SSC-A15 (manufactured by Shinano Electric Refining Co., Ltd.)) as thermally conductive particles.

### <2> Production, Measurement, and Evaluation of Backing Material Sheet

The composition for a backing material layer prepared as described above was poured into a square mold having one side of 30 mm and a desired depth and cured by heating at 80° C. for 18 hours and then at 150° C. for 1 hour to produce a square backing material sheet having one side of 30 mm and a desired thickness. The depth of the mold used and the thickness of the obtained sheet are 2 mm or 0.5 mm, respectively. The following measurements and evaluations were performed on the backing material sheet.

### (1) Measurement of Thermal Conductivity

The square backing material sheet having a thickness of 0.5 mm was cut into a strip shape having a width of 5 mm to prepare a test piece. The prepared test piece was measured by a laser flash method according to Japanese Industrial Standards (JIS) R 1611. The test piece formed of any of the compositions for a backing material layer exhibited a good value of a thermal conductivity of 1.0 W/m·K.

### (2) Measurement of Attenuation Rate

An intensity of a reflected echo was measured using a sing-around type acoustic velocity measurement apparatus (manufactured by Ultrasonic Engineering Co., Ltd., trade name “UVM-2 type”) based on a method described in a method of measuring an ultrasonic attenuation coefficient of a solid according to Japanese Industrial Standards (JIS) Z 2354 (2012). In the measurement, a measurement probe of 2 MHz was used in water at 25° C., and an attenuation rate was obtained from a difference in intensity of the reflected echo depending on the presence or absence of a measurement test piece used for measuring the acoustic velocity and a thickness of the measurement test piece. The test piece formed of any of the compositions for a backing material layer exhibited a good value of an attenuation rate of 4.0 dB/mm. MHz or more.

The above results show that the polyurea resin, the epoxy resin having a polyurethane structure, or the epoxy resin having a polyetheramine structure is a suitable material as a backing material layer of an ultrasonic endoscope, because these resins exhibit good thermal conductivity and attenuation rate.

The ultrasonic endoscope 12 has a configuration in which the imaging module penetrates the inside of the metal ring 41c, and the endoscope observation part 38 is located on the distal end side with respect to the ultrasonic observation part 36, but the present disclosure is not limited thereto. For example, the endoscope observation part 38 may be disposed on the proximal end side with respect to the ultrasonic observation part 36, and a front side of the distal end part 40 may be imaged from a lateral side of the distal end part 40.

As described above, at least the following matters are described in the present specification.

(1)

An ultrasonic endoscope comprising: a tubular member that is configured to include a metal; an ultrasonic oscillator that is provided along an outer peripheral surface of the tubular member; and a backing material layer that is provided between the ultrasonic oscillator and the tubular member, in which the backing material layer is configured to include at least one kind of a polyurea resin, an epoxy resin having a polyurethane structure, or an epoxy resin having a polyetheramine structure.

(2)

The ultrasonic endoscope according to (1), in which the backing material layer is configured to include a heat radiation filler.

(3)

The ultrasonic endoscope according to (2), in which a thermal conductivity of the heat radiation filler is 30 W/m·K or more.

(4)

The ultrasonic endoscope according to (3), in which the heat radiation filler includes at least one of aluminum oxide, tungsten oxide, silicon carbide, tungsten carbide, silicon nitride, boron nitride, or aluminum nitride.

(5)

The ultrasonic endoscope according to any one of (1) to (4), in which the tubular member and the backing material layer are fixed via an interlayer.

(6)

The ultrasonic endoscope according to (5), in which the interlayer prevents ions generated from the metal included in the tubular member from coming into contact with the backing material layer.

(7)

The ultrasonic endoscope according to (6), in which the backing material layer is fixed to the interlayer formed on the outer peripheral surface of the tubular member.

(8)

The ultrasonic endoscope according to any one of (1) to (7), further comprising: an accommodation portion that accommodates the tubular member; and a filler that fills a periphery of the tubular member in the accommodation portion, in which the filler has a viscosity of less than 100 Pa·sec at a shear rate of 0.01/s in a non-cured state.

(9)

The ultrasonic endoscope according to (8), in which the filler is configured to include a resin as a base material and a thixotropic agent.

(10)

The ultrasonic endoscope according to (9), in which the thixotropic agent includes fumed silica.

(11)

The ultrasonic endoscope according to any one of (1) to (10), in which a thickness of the backing material layer is 0.5 mm or more and 1.5 mm or less.

(12)

The ultrasonic endoscope according to any one of (1) to (11), in which a vibration frequency of the ultrasonic oscillator has a center frequency of 5 MHz or more and 12 MHz or less.

(13)

The ultrasonic endoscope according to any one of (1) to (12), further comprising: an imaging module including an imaging element, in which the imaging module is inserted into the tubular member.

### EXPLANATION OF REFERENCES

10: endoscope apparatus
12: ultrasonic endoscope
14: ultrasound processor device
16: endoscope processor device
18: light source device
20: monitor
21a: water supply tank
21b: suction pump
22: insertion part
24: operating part
26: universal cord
28a: air/water supply button
28b: suction button
29: angle knob
30: treatment tool insertion port
32A, 32B, 32C: connector
34a: air/water supply tube
34b: suction tube
36: ultrasonic observation part
38: endoscope observation part
40: distal end part
41a: distal end component
41b: proximal end-side ring
41c: metal ring
42: bendable part
44: soft part
46: ultrasound transmission/reception section
48: ultrasonic oscillator
52a: individual electrode
52b: common electrode
52: electrode part
53: interlayer
54: backing material layer
55: filler
56: flexible printed substrate
58: coaxial cable
64: acoustic matching layer
66: acoustic lens
72: signal line bundle
76: treatment tool outlet port
78: observation window
80: illumination window
82: cleaning nozzle
85: observation system unit
86: objective lens
88: prism
90: imaging element
92: substrate
94: signal cable
98: light guide
100: air/water supply channel
110: bracket support member
120: bracket

What is claimed is:

1. An ultrasonic endoscope comprising:
a tubular member comprising a metal;
an ultrasonic oscillator that is provided along an outer peripheral surface of the tubular member; and
a backing material layer that is provided between the ultrasonic oscillator and the tubular member,
wherein the backing material layer comprising at least one kind of a polyurea resin, an epoxy resin having a polyurethane structure, or an epoxy resin having a polyetheramine structure.

2. The ultrasonic endoscope according to claim 1,
wherein the backing material layer comprises a heat radiation filler.

3. The ultrasonic endoscope according to claim 2,
wherein a thermal conductivity of the heat radiation filler is 30 W/m·K or more.

4. The ultrasonic endoscope according to claim 3,
wherein the heat radiation filler comprises at least one of: aluminum oxide, tungsten oxide, silicon carbide, tungsten carbide, silicon nitride, boron nitride, or aluminum nitride.

5. The ultrasonic endoscope according to claim 1,
wherein the tubular member and the backing material layer are fixed with each other via an interlayer.

6. The ultrasonic endoscope according to claim 2,
wherein the tubular member and the backing material layer are fixed with each other via an interlayer.

7. The ultrasonic endoscope according to claim 3,
wherein the tubular member and the backing material layer are fixed with each other via an interlayer.

8. The ultrasonic endoscope according to claim 4,
wherein the tubular member and the backing material layer are fixed with each other via an interlayer.

9. The ultrasonic endoscope according to claim 5,
wherein the interlayer prevents ions generated from the metal contained in the tubular member from coming into contact with the backing material layer.

10. The ultrasonic endoscope according to claim 6,
wherein the interlayer prevents ions generated from the metal contained in the tubular member from coming into contact with the backing material layer.

11. The ultrasonic endoscope according to claim 7,
wherein the interlayer prevents ions generated from the metal contained in the tubular member from coming into contact with the backing material layer.

12. The ultrasonic endoscope according to claim 8,
wherein the interlayer prevents ions generated from the metal contained in the tubular member from coming into contact with the backing material layer.

13. The ultrasonic endoscope according to claim 9,
wherein the backing material layer is fixed to the interlayer formed on the outer peripheral surface of the tubular member.

14. The ultrasonic endoscope according to claim 1, further comprising:
an accommodation portion that accommodates the tubular member; and
a filler that fills a periphery of the tubular member in the accommodation portion, wherein the filler has a viscosity of less than 100 Pa·sec at a shear rate of 0.01/s in a non-cured state.

15. The ultrasonic endoscope according to claim 2, further comprising:
an accommodation portion that accommodates the tubular member; and
a filler that fills a periphery of the tubular member in the accommodation portion,
wherein the filler has a viscosity of less than 100 Pa·sec at a shear rate of 0.01/s in a non-cured state.

16. The ultrasonic endoscope according to claim 14,
wherein the filler is configured to contain a resin as a base material, and a thixotropic agent.

17. The ultrasonic endoscope according to claim 16,
wherein the thixotropic agent comprises fumed silica.

18. The ultrasonic endoscope according to claim 1,
wherein a thickness of the backing material layer is 0.5 mm or more and 1.5 mm or less.

19. The ultrasonic endoscope according to claim 18,
wherein a vibration frequency of the ultrasonic oscillator has a center frequency of 5 MHz or more and 12 MHz or less.

20. The ultrasonic endoscope according to claim 1, further comprising:
an imaging module including an imaging element,
wherein the imaging module is inserted into the tubular member.
